(19) United States
(12) Patent Application Publication
(10) Pub. No.: US 2022/0031446 A1
(43) Pub. Date: Feb. 3, 2022

(54) ARTIFICIAL DERMIS REPAIR MATERIAL AND PREPARATION METHOD THEREFOR

(71) Applicant: SHENZHEN TSINGCARE MEDICAL INSTRUMENTS CO., LTD., Shenzhen (CN)

(72) Inventors: Danyan WANG, Shenzhen (CN); Rongwei TAN, Shenzhen (CN); Zhending SHE, Shenzhen (CN); Yuanjun GUO, Shenzhen (CN); Xi LIU, Shenzhen (CN); Mengqiang XU, Shenzhen (CN); Yingying CHEN, Shenzhen (CN)

(21) Appl. No.: 17/275,960

(22) PCT Filed: Oct. 12, 2018
(86) PCT No.: PCT/CN2018/110016

§ 371 Date: Mar. 12, 2021

(30) Foreign Application Priority Data
Sep. 19, 2018 (CN) 201811096057.0

(51) Int. Cl. A61F 2/10; A61L 27/60; A61L 27/16; A61L 27/24; A61L 27/26; A61L 27/56; A61L 27/58

(52) U.S. Cl. CPC A61F 2/105; A61L 27/60; A61L 27/165; A61L 27/24; A61L 2300/404; A61L 27/56; A61L 27/58; A61F 2210/0004; A61L 27/26

(57) ABSTRACT

An artificial dermis repair material and a preparation method therefor. The artificial dermis repair material comprises a silicon rubber layer and a collagen complex layer adhered to each other; the collagen complex layer is prepared from raw materials comprising collagen, a mucopolysaccharide, and an antibacterial agent, which undergo a crosslinking reaction. The silicon rubber layer has high strength, high elasticity and softness, is easy to stitch, and has a good fitting performance; the collagen complex layer has good biocompatibility, degradability and an antibacterial property, and is suitable for repair and reconstruction of a dermal tissue.

### TECHNICAL FIELD

The present disclosure relates to the field of medical instruments and the field of tissue engineering regeneration, and in particular, to an artificial dermal repair material and a preparation method thereof.

### BACKGROUND

Skin is the largest organ of a human body, and can protect the body from harmful substances from outside, prevent the loss of water, electrolytes, and the like in the body, maintain the stability of an internal environment of the human body, and participate in immune and metabolic processes of the human body. Skin tissue defects caused by burns, diseases, physical trauma, wars, and the like affect the quality of life of people and even threaten lives. According to a report by the World Health Organization (WHO) (January 2018), 180,000 people die of burns worldwide each year. In 2004, nearly 11 million people worldwide were severely burned and required medical attention. In 2000, direct medical expenditure on burns for children in America exceeded $211 million. In 2007, hospital burn management expenditure in Norway exceeded €10.5 million. Indirect losses such as lost wages, long-term care for disabilities and psychological trauma, and invested household resources have also exacerbated the impact on society and economy. When the skin is damaged in a large area or severely, the skin cannot repair itself and needs to be repaired by skin grafts/skin flaps or dermal substitute transplantation. At present, dermal substitutes for treating a wound surface mainly include artificial dermis (made of biological and synthetic materials), allogeneic or xenogeneic natural dermis (treated by decellularization), tissue engineering active dermis (such as Apligraf®, IERFU®), etc. Artificial dermal substitute products have no limitation on material sources and ethics, have low virus infection risk, are easy to store and transport, can be produced in batches, have increasingly mature production, safety, and quality control, and have low cost, thereby gradually occupying the mainstream of market development.

Collagen, as the most commonly used biological material, is widely used to prepare artificial skin. For example, both Integra® and Lando® artificial dermis that have been approved for marketing use collagen-glycosaminoglycan (GAG) materials and are clinically used for deep burns and full-thickness skin defects. US Patent US 20160143726 A1 discloses a method of preparing a collagen-GAG-elastin scaffold, and the preparation process thereof includes preparing a collagen-GAG-elastin complex solution, freeze-drying the complex solution into a porous scaffold, and then performing cross-linking. These scaffolds can be used in the medical and surgical fields, such as the reconstruction of dermal tissue. However, existing artificial dermal substitutes still cannot meet the requirements of ideal dermal substitutes, and have problems such as vascularization, wound infection, and the like in clinical applications. Therefore, it is necessary to invent materials with better physical properties and antibacterial properties for use in the field of skin tissue engineering.

### SUMMARY

A technical problem to be solved by the present disclosure is to provide an artificial dermal repair material and a preparation method thereof. The prepared artificial dermal repair material has good physical properties and antibacterial properties, and is applicable in the field of skin tissue engineering.

The present disclosure adopts technical solutions as follows.

The present disclosure provides an artificial dermal repair material, which includes a silicone rubber layer and a collagen complex layer bonded to each other. The collagen complex layer is prepared by raw materials including collagen, glycosaminoglycan, and an antibacterial agent by a cross-linking reaction.

In some preferred embodiments, the silicone rubber layer includes a first silicone rubber layer, a second mesh-shaped silicone rubber intermediate layer, and a third silicone rubber layer which are provided in sequence. A tensile strength of the second mesh-shaped silicone rubber intermediate layer is greater than a tensile strength of the first silicone rubber layer and a tensile strength of the third silicone rubber layer.

In some more preferred embodiments, the tensile strength of the first silicone rubber layer and/or the third silicone rubber layer is less than 2 MPa. The tensile strength of the second mesh-shaped silicone rubber intermediate layer is greater than 10 MPa.

In some preferred embodiments, the collagen complex layer is of a gradient porous structure. Further, sizes of holes in the gradient porous structure are distributed in a gradient manner, and a hole size increases from 50 μm to 250 μm.

In some preferred embodiments, the raw materials include 73% to 97% of the collagen, 2% to 15% of the glycosaminoglycan, and 1% to 12% of the antibacterial agent.

In some preferred embodiments, the raw materials include 78% to 93% of the collagen, 3% to 10% of the glycosaminoglycan, and 4% to 12% of the antibacterial agent.

In some preferred embodiments, the glycosaminoglycan is chondroitin sulfate or hyaluronic acid.

In some preferred embodiments, the antibacterial agent is at least one selected from the group consisting of polyhexamethylene biguanide (PHMB), lysozyme, and lysostaphin.

In some preferred embodiments, at least one of the silicone rubber layer and the collagen complex layer is provided with a plurality of holes.

In some more preferred embodiments, dimeters of the holes on the silicone rubber layer are in a range of 50 μm to 1000 μm. Spacing of the holes are in a range of 0.5 mm to 10 mm.

In some more preferred embodiments, dimeters of the holes on the collagen complex layer are in a range of 50 μm to 500 μm. Spacing of the holes are in a range of 0.5 mm to 5 mm.

In some preferred embodiments, the collagen complex layer has a thickness of 1 mm to 3 mm.

In some preferred embodiments, the silicone rubber layer has a thickness of 0.2 mm to 0.3 mm.

The present disclosure further provides a method of preparing the artificial dermal repair material as described above, which includes:

preparing a collagen complex layer, including:

dissolving glycosaminoglycan in a solvent to obtain a glycosaminoglycan solution, and dissolving collagen in a solvent to obtain a collagen solution;

mixing the glycosaminoglycan solution and the collagen solution, performing centrifugation and collecting a precipitate, and redissolving the precipitate by adding acetic acid to obtain a collagen-glycosaminoglycan complex solution; and

adding a cross-linking agent and an antibacterial agent into the collagen-glycosaminoglycan complex solution, performing freeze-drying, and then further performing a cross-linking reaction under a vacuum condition, to obtain the collagen complex layer;

preparing a silicone rubber layer,

bonding the collagen complex layer and the silicone rubber layer, and

optionally preparing a hole on at least one of the collagen complex layer and the silicone rubber layer.

In some preferred embodiments, the cross-linking reaction is further performed under a high-temperature vacuum condition of 100° C. to 120° C.

In some preferred embodiments, the preparing the silicone rubber layer includes: mixing and curing materials of a first silicone rubber to form the first silicone rubber layer; mixing materials of a second silicone rubber to form a mixed solution, preparing a mesh-shaped layer on the first silicone rubber layer with the mixed solution by using a dispenser, and performing curing to form a second mesh-shaped silicone rubber intermediate layer; and leveling materials of a third silicone rubber on a surface of the second mesh-shaped silicone rubber intermediate layer, and performing curing to form a third silicone rubber layer. A tensile strength of the materials of the second silicone rubber after film formation is greater than a tensile strength of the materials of the first silicone rubber and a tensile strength of the materials of the third silicone rubber after film formation.

In some preferred implementations, the acetic acid has a concentration of 0.03M to 0.1M.

In some preferred embodiments, the performing freeze-drying includes: performing freezing under a low temperature environment, annealing for 5 min to 10 min, and then freezing under a low temperature environment, and then freeze-drying in a freeze dryer.

In further preferred embodiments, the low temperature environment has a temperature in a range of −50° C. to 70° C.

In some preferred embodiments, the cross-linking agent is one selected from the group consisting of glutaraldehyde, genipin, and carbodiimide.

In some preferred embodiments, the cross-linking agent accounts for the collagen-glycosaminoglycan complex solution in a mass range of 0.005% to 0.015%.

In some preferred embodiments, the glycosaminoglycan solution is added to the collagen solution for mixing by high-speed airflow atomization.

In some preferred embodiments, the holes may be prepared by any of laser drilling, physical puncturing, and freeze-drying into holes in a special mold with regularly arranged protrusions at a bottom thereof.

In some preferred embodiments, the collagen complex layer and the silicone rubber layer are bonded together by silicone gel.

The beneficial effects achieved by the present disclosure are:

In the artificial dermal repair material provided by the present disclosure, the silicone rubber layer has high strength, high elasticity, and softness, is easy stitch, and has good fitting performance. The collagen complex layer is beneficial to the growth of fibroblasts, endothelial cells, and capillaries, and has good biocompatibility, degradability, and antibacterial property, which is applicable for the repair and reconstruction of dermal tissue.

### BRIEF DESCRIPTION OF THE DRAWINGS

FIG. 1 is an atomic force microscope (AFM) image of a collagen-chondroitin sulfate complex in Example 1.

FIG. 2 is a scanning electron microscope (SEM) image of the collagen-chondroitin sulfate complex in Example 1.

FIG. 3 is a SEM image showing a cross section of a collagen complex layer in Example 1.

FIG. 4 is a SEM image of a drilled collagen complex layer in Example 2.

FIG. 5 is photos showing results of bacteriostatic experiments in Example 3.

FIG. 6 is a photo showing a drilled silicone rubber layer in Example 4.

### DETAILED DESCRIPTION OF THE EMBODIMENTS

In the following, the concept and the produced technical effects of the present disclosure will be clearly and completely described in conjunction with the embodiments to fully understand the purpose, features and effects of the present disclosure. Apparently, the described embodiments are only a part of the embodiments of the present disclosure, rather than all of them. Based on the embodiments of the present disclosure, other embodiments obtained by those skilled in the art without creative work shall fall within the protection scope of the present disclosure.

### Example 1

This example provides an artificial dermal repair material, and a preparation process thereof is as follows.

At S1, a collagen complex layer was prepared.

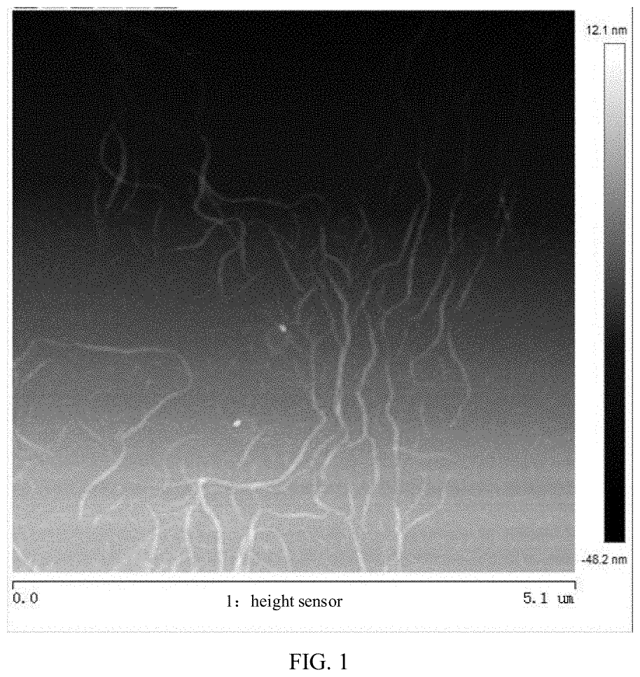

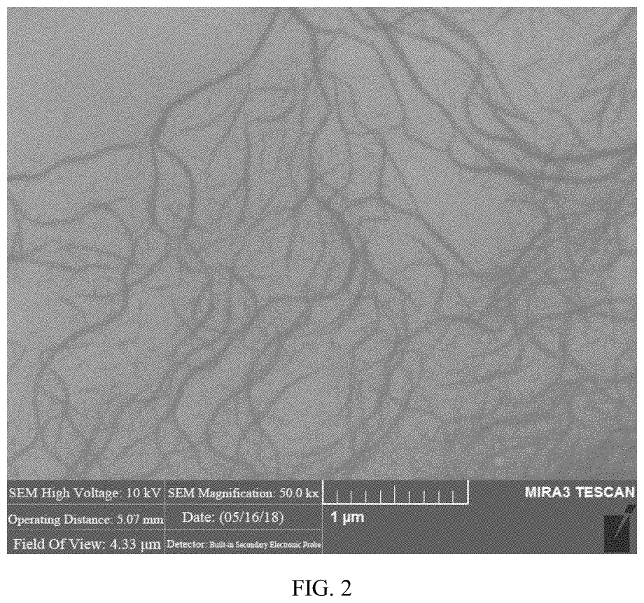

Collagen was weighed and dissolved in water to prepare 200 g of a collagen solution with a mass concentration of 0.3%. Then, 0.056 g of chondroitin sulfate was weighed and dissolved in water to prepare a chondroitin sulfate solution with a mass concentration of 1.7 mg/mL. The chondroitin sulfate solution was atomized by high-speed airflow and then added to the collagen solution, and continuously stirred for 2 hours to obtain a collagen-chondroitin sulfate complex. The collagen-chondroitin sulfate complex was centrifuged at 8000 rpm for 15 minutes. The precipitate was collected, and 0.05M acetic acid was added to re-dissolve the precipitate to make the concentration of collagen 0.9% (w/w), and then stirred for 2.5 h to be dispersed uniformly, thereby forming a dispersion of the collagen-chondroitin sulfate complex. The dispersion of the collagen-chondroitin sulfate complex was characterized. An AFM image was as shown in FIG. 1 and a SEM image was as shown in FIG. 2. It can be seen from the figures that the obtained complex can be co-precipitated to form nano-scale collagen fibers, and the nano-scale collagen fibers can be self-assembled to form collagen fiber bundles, forming a uniformly dispersed collagen-chondroitin sulfate complex. A glutaraldehyde crosslinking agent solution (acetic acid as solvent) was added to make the concentration of the collagen 0.7% (w/w), where a mass fraction of the added glutaraldehyde was 0.01% (that is, a mass fraction of the glutaraldehyde accounting for the collagen complex solution), and then stirred for 5 h to be uniformly distributed. Then, a polyhexamethylene biguanide (PHMB) antibacterial agent solution was added to make the concentration of the collagen 0.65% (w/w), where the amount of the added PHMB was 0.028 g, and then was stirred for 2 h to be uniformly distributed, and then was poured into an aluminum alloy mold, leveled, pre-frozen at −60° C. for 2 h, and then annealed for 5 min to 10 min, and then frozen at −60° C. for half an hour, and then transferred to a freeze dryer for freeze-drying for 24 h, and then placed under a high-temperature vacuum condition of 105° C. for high-temperature cross-linking for 24 hours, to obtain the collagen complex layer.

At S2, a silicone rubber layer was prepared.

Components A and B of liquid silicone rubber with normal strength (with tensile strength <2 Mpa after film formation) were mixed together in a ratio of 1:1, stirred at 300 rpm for 10 minutes, and then defoamed, and then poured into a polytetrafluoroethylene mold, leveled, and placed in a drying oven and cured at 150° C. for 2 hours to obtain a first silicone rubber layer with a thickness of 0.12 mm.

Components A and B of liquid silicone rubber with high strength (with tensile strength >10 Mpa after film formation) were mixed together in a ratio of 1:1, stirred at 300 rpm for 10 minutes to form a mixed solution, and then the mixed solution was defoamed. A layer of silicone rubber layer in a parallelogram mesh shape was uniformly prepared on a surface of the first silicone rubber layer with the mixed solution by using a dispenser, in which a spacing between parallel lines was 1 mm to 10 mm, and then was placed in a drying oven for curing, and cured at 150° C. for 2 hours, to obtain a second mesh-shaped silicone rubber layer with a thickness of 0.02 mm.

Then, a liquid silicone rubber with normal strength (with tensile strength <2 Mpa after film formation) was leveled on a surface of the second mesh-shaped silicone rubber layer, and then placed in a drying oven, and cured at 150° C. for 2 hours, to obtain a third silicone rubber layer with a thickness of 0.12 mm. Finally, a silicone rubber layer with a thickness of 0.26 mm and having a mesh-shaped intermediate layer was formed.

At S3, a layer of silicone gel was coated on a surface of the silicone rubber layer, and a drilled collagen complex layer was bonded to a surface of the silicone rubber film to obtain the artificial dermal repair material.

At S4, packaging and sterilizing were performed. The prepared artificial dermal repair material was packaged, and sterilized by high-energy electron beam with a sterilization dose of 15 KGy to 20 KGy.

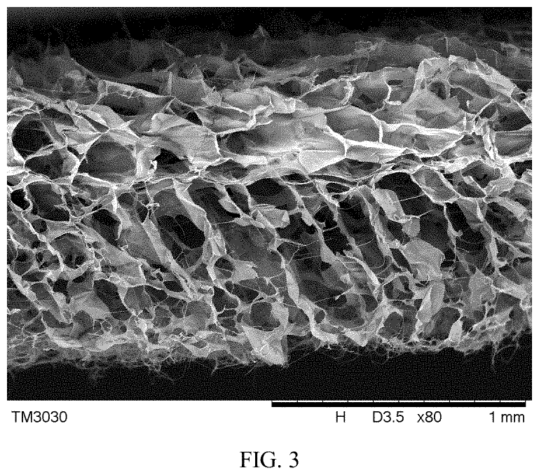

The collagen complex layer prepared in this example is characterized, and a SEM image showing a cross section of the collagen complex layer is as shown in FIG. 3. It can be seen from the figure that the prepared collagen complex layer has a three-dimensional gradient porous structure, and a pore size increases from 50 μm to 250 μm from a position in contact with the silicone rubber layer to a position away from the silicone rubber layer. In the prior art, the gradient porous structure is prepared generally by spraying and freeze-drying multiple layers of mixed solutions with different concentrations to achieve the effect of gradient pores, which has a more complicated preparation process, and the pore size increases suddenly. In the present disclosure, the gradient pores are prepared by pre-freezing, annealing, and freeze-drying, the operation is simple, the pore size increases transitionally and naturally, which is more beneficial to the growth of fibroblasts, endothelial cells, and capillaries.

The silicone rubber layer prepared in this implementation is measured for tensile strength. The specific steps of the measurement are as follows. Referring to the standard of GB/T 1040.3-2006 “Measurement of Tensile Properties of Plastics Part 3: Test Conditions for Films and Sheets”, type 5 dumbbell-shaped test pieces are prepared by a cutter, respectively, and 3 parallel test pieces are prepared for each group of samples. A specified marking is marked on the test piece. A thickness h and a width b of each test piece are measured within 5 mm from each end of the gauge length in the middle of each test piece. 3 points are measured to calculate the average value. The electronic universal testing machine is turned on, test parameters are set, and related test parameters such as thickness and gauge length are input. The test speed was set to 500 mm/min. The sensor is 1000 N. The test pieces are mounted on the fixture so that long axes of the test pieces are in line with an axis of the testing machine. The run button is clicked to test. The tensile strength is calculated according to the following formula.

Tensile strength calculation formula: σM=F/S. In the formula: GM: tensile strength in MPa; F: force (maximum value) required for tearing the test piece, in N; S: original cross-sectional area of the test piece, mm². The test results are shown in Table 1.

TABLE 1
The tensile strength of the silicone rubber layer in Example 1

| Sample number | Tensile strength (MPa) |
|---|---|
| 1 | 9.2 |
| 2 | 9.3 |
| 3 | 9.5 |

The test results show that the silicone rubber layer with the mesh-shaped intermediate layer provided by the present disclosure has excellent mechanical strength. The mesh-shaped intermediate layer is similar to a layer of adjustable reinforcing mesh, which can balance the high strength and flexibility of the entire silicone rubber layer, improve the mechanical properties of the silicone rubber layer, so that the silicone rubber layer has the appropriate fitting performance and good tensile and tearing strength, and is convenient for stitching, and has a close attachment to a wound surface.

### Example 2

This example provides an artificial dermal repair material, and a preparation process thereof is as follows.

At S1, a collagen complex layer was prepared.

Collagen was weighed and dissolved in water to prepare 200 g of a collagen solution with a mass concentration of 0.3%. Then, 0.013 g of chondroitin sulfate was weighed and dissolved in water to prepare a chondroitin sulfate solution with a mass concentration of 0.37 mg/mL. The chondroitin sulfate solution was atomized by high-speed airflow and then added to the collagen solution, and continuously stirred for 2 hours to obtain a collagen-chondroitin sulfate complex. The collagen-chondroitin sulfate complex was centrifuged at 8000 rpm for 15 minutes. The precipitate was collected, and 0.03M acetic acid was added to re-dissolve the precipitate, to make the concentration of collagen 0.9% (w/w), and then stirred for 2.5 h to be dispersed uniformly. Then, an acetic acid solution of glutaraldehyde was added to form a collagen complex solution to make the concentration of the collagen 0.7% (w/w), where a mass fraction of the added glutaraldehyde was 0.005% (that is, a mass fraction of the glutaraldehyde accounting for the collagen complex solution), and then stirred for 5 h to be uniformly distributed. Then, a PHMB antibacterial agent solution was added to make the concentration of the collagen 0.65% (w/w), where the amount of the added PHMB was 0.0062 g, and then was stirred for 2 h to be uniformly distributed, and then was poured into an aluminum alloy mold, leveled, pre-frozen at −60° C. for 2 h, and then annealed for 5 min to 10 min, and then frozen at −60° C. for half an hour, and then transferred to a freeze dryer for freeze-drying for 24 h, and then placed under a high-temperature vacuum condition of 105° C. for high-temperature cross-linking for 24 hours, to obtain the collagen complex layer.

At S2, a silicone rubber layer was prepared.

Components A and B of liquid silicone rubber with normal strength (with tensile strength <2 Mpa after film formation) were mixed together in a ratio of 1:1, stirred at 300 rpm for 10 minutes, and then defoamed, and then poured into a polytetrafluoroethylene mold, leveled, and placed in a drying oven and cured at 150° C. for 2 hours to obtain a first silicone rubber layer with a thickness of 0.1 mm.

Components A and B of liquid silicone rubber with high strength (with tensile strength >10 Mpa after film formation) were mixed together in a ratio of 1:1, stirred at 300 rpm for 10 minutes to form a mixed solution, and then the mixed solution was defoamed. A layer of silicone rubber layer in a parallelogram mesh-shape was uniformly prepared on a surface of the first silicone rubber layer with the mixed solution by using a dispenser, in which a spacing between parallel lines was 1 mm to 10 mm, and then was placed in a drying oven for curing and cured at 150° C. for 2 hours, to obtain a second mesh-shaped silicone rubber layer with a thickness of 0.01 mm.

Then, a liquid silicone rubber with normal-strength (with tensile strength <2 Mpa after film formation) was leveled on a surface of the second mesh-shaped silicone rubber layer, and then placed in a drying oven, and cured at 150° C. for 2 hours, to obtain a third silicone rubber layer with a thickness of 0.1 mm. Finally, a silicone rubber layer with a thickness of 0.21 mm and having a mesh-shaped intermediate layer was formed.

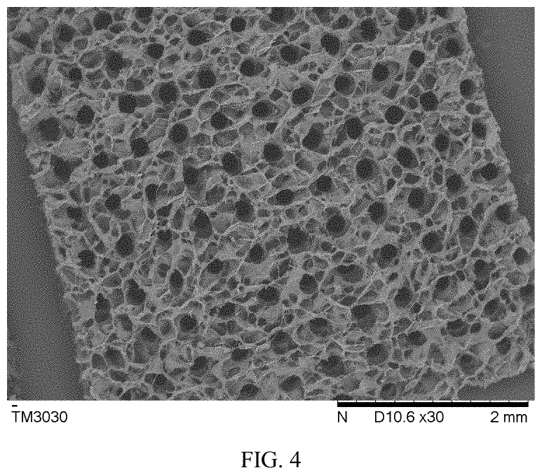

At S3, parameters such as hole spacing, number of rows, and hole size were set on an operation interface of the laser drilling machine. The collagen complex layer obtained in step S1 was placed and fixed in the machine, and the program was run to drill holes. The hole size was 250 The hole spacing was 0.5 mm. The drilled collagen complex layer was characterized, and a SEM image thereof was as shown in FIG. 4.

At S4, a layer of silicone gel was coated on a surface of the silicone rubber layer, and the drilled collagen complex layer was bonded to a surface of the silicone rubber layer to obtain the artificial dermal repair material.

At S5, packaging and sterilizing were performed. The prepared artificial dermal repair material was packaged and sterilized by high-energy electron beam, with a sterilization dose of 15 KGy to 20 KGy.

### Example 3

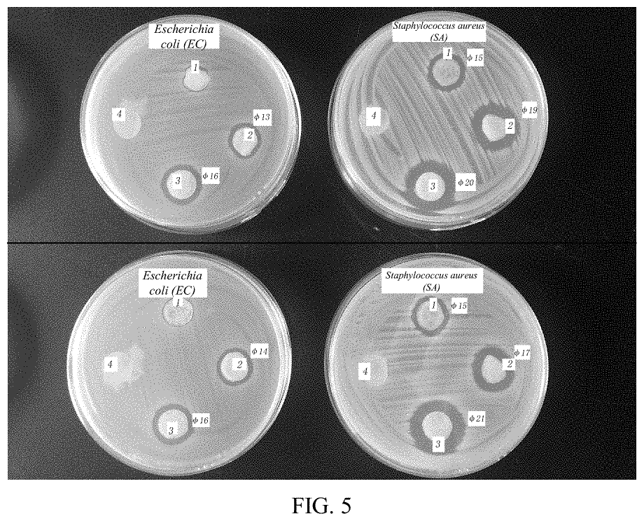

This example provides three types of artificial dermal repair materials, which are the same as that in Example 1. The difference was in that a content of the PHMB was 1.2%, 6%, and 12%, respectively. Bacteriostatic experiments were carried out on the three types of artificial dermal repair materials, and the operation was as follows: the artificial dermal repair materials were cut into discs with a diameter of 12 cm, and sterilized by ultraviolet irradiation for 30 minutes in a super clean bench for standby. A Mueller-Hinton (MH) agar medium was prepared, and then heated to be dissolved completely, and then sterilized at 121° C. for 15 minutes, followed by being poured into a dish in a sterile environment when the temperature dropped to 40° C. to 50° C., and then cooled and solidified for later use. A sterile cotton swab was used to dip a corrected Escherichia coli and Staphylococcus aureus bacterial solutions at 0.5 Mcfarland (one Mcfarland=3*10⁸ cfu/ml), and then the excess bacterial solution was squeezed off by slightly abutting against a tube wall. The entire surface of the medium was coated by the cotton swab, the plate was rotated by 60 degrees each time, the smearing was carried out three times, and finally, was wiped around a periphery for two circles, to ensure that the bacterial solution was uniformly smeared. The sample was attached on a surface of the plate by sterile forceps, and cannot be taken away after being attached. Four samples were attached to each dish. The plate was turned over and cultured in a 37° C. constant temperature incubator for 18 hours to 24 hours, and then taken out for measurement. A diameter of an inhibition zone was measured by a ruler on a back of the plate (measured crosswise, horizontally and vertically, calculating the average value, and making the average value an integer). An edge of the inhibition zone was limited to the fact that no obvious growth of bacteria was visible to the naked eye. The results were as shown in FIG. 5. In the figure, the mark E indicates the Escherichia coli bacteriostatic experiment, and the mark S indicates the Staphylococcus aureus bacteriostatic experiment. Nos. 1 to 3 correspond to artificial dermal repair materials with a PHMB content of 1.2%, 6%, and 12%, respectively. No. 4 is a blank control group without PHMB. The experimental results show that the artificial dermal repair materials prepared by the present disclosure have bacteriostasis to both Gram-negative bacteria (such as Escherichia coli) and Gram-positive bacteria (such as Staphylococcus aureus), and have stronger bacteriostasis to Gram-positive bacteria.

### Example 4

This example provides an artificial dermal repair material, and a preparation process thereof is as follows.

At S1, a collagen complex layer was prepared.

Collagen was weighed and dissolved in water to prepare 200 g of a collagen solution with a mass concentration of 0.3%. Then, 0.123 g of chondroitin sulfate was weighed and dissolved in water to prepare a chondroitin sulfate solution with a mass concentration of 3.54 mg/mL. The chondroitin sulfate solution was atomized by high-speed airflow and then added to the collagen solution, and continuously stirred for 2 hours to obtain a collagen-chondroitin sulfate complex. The collagen-chondroitin sulfate complex was centrifuged at 8000 rpm for 15 minutes. The precipitate was collected, and 0.1M acetic acid was added to re-dissolve the precipitate to make the concentration of collagen 0.9% (w/w), and then stirred for 2.5 h to be dispersed uniformly. Then, an acetic acid solution of glutaraldehyde was added to form a collagen complex solution to make the concentration of the collagen 0.7% (w/w), where a mass fraction of the added glutaraldehyde was 0.015% (that is, a mass fraction of the glutaraldehyde accounting for the collagen complex solution), and then stirred for 5 h to be uniformly distributed. Then, a PHMB antibacterial agent solution was added to make the concentration of the collagen 0.65% (w/w), where the amount of the added PHMB was 0.099 g, and then was stirred for 2 h to be uniformly distributed, and then was poured into an aluminum alloy mold, leveled, and pre-frozen at −60° C. for 2 h, and then annealed for 5 min to 10 min, and then frozen at −60° C. for half an hour, and then transferred to a freeze dryer for freeze-drying for 24 h, and then placed under a high-temperature vacuum condition of 105° C. for high-temperature cross-linking for 24 hours, to obtain the collagen complex layer.

At S2, a silicone rubber layer was prepared.

Components A and B of liquid silicone rubber with normal strength (with tensile strength <2 Mpa after film formation) were mixed together in a ratio of 1:1, stirred at 300 rpm for 10 minutes, and then defoamed, and then poured into a polytetrafluoroethylene mold, leveled, and placed in a drying oven and cured at 150° C. for 2 hours to obtain a first silicone rubber layer with a thickness of 0.15 mm.

Components A and B of liquid silicone rubber with high strength (with tensile strength >10 Mpa after film formation) were mixed together in a ratio of 1:1, stirred at 300 rpm for 10 minutes to form a mixed solution, and then the mixed solution was defoamed. A layer of silicone rubber layer in a parallelogram mesh-shape was uniformly prepared on a surface of the first silicone rubber layer with the mixed solution by using a dispenser, in which a spacing between parallel lines was 1 mm to 10 mm, and then was placed in a drying oven for curing and cured at 150° C. for 2 hours, to obtain a second mesh-shaped silicone rubber layer with a thickness of 0.05 mm.

Then, a liquid silicone rubber with normal-strength (with tensile strength <2 Mpa after film formation) was leveled on a surface of the second mesh-shaped silicone rubber layer, and then placed in a drying oven, and cured at 150° C. for 2 hours, to obtain a third silicone rubber layer with a thickness of 0.1 mm. Finally, a silicone rubber layer with a thickness of 0.3 mm and having a mesh-shaped intermediate layer was formed.

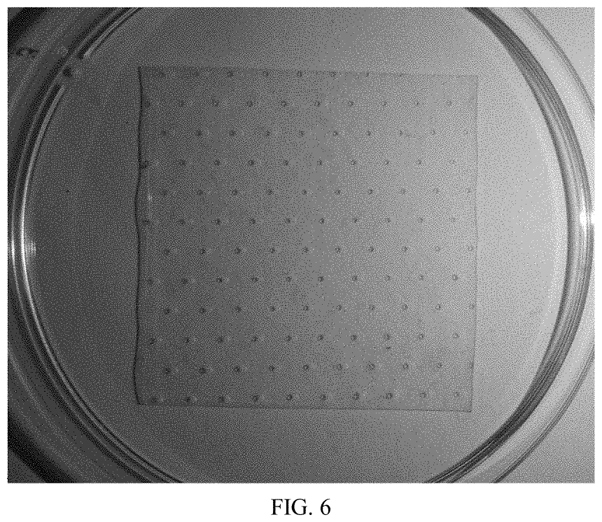

At S3, parameters such as hole spacing, number of rows, and hole size were set on the operation interface of the laser drilling machine. The collagen complex layer obtained in step S1 was placed and fixed in the machine, and the program was run to drill holes. The hole size was 250 The hole spacing was 5 mm. The silicone rubber layer obtained in step S2 was drilled, with a hole size of 500 μm and a hole spacing is 5 mm. The drilled silicone rubber layer was as shown in FIG. 6.

At S4, a layer of silicone gel was coated on a surface of the silicone rubber layer, and the drilled collagen complex layer was bonded to a surface of the drilled silicone rubber layer to obtain the artificial dermal repair material.

At S5, packaging and sterilizing were performed. The prepared artificial dermal repair material was packaged and sterilized by high-energy electron beam, with a sterilization dose of 15 KGy to 20 KGy.

1. An artificial dermal repair material, comprising a silicone rubber layer and a collagen complex layer bonded to each other, the collagen complex layer being prepared by raw materials comprising collagen, glycosaminoglycan, and an antibacterial agent by a cross-linking reaction.

2. The artificial dermal repair material according to claim 1, wherein the silicone rubber layer comprises a first silicone rubber layer, a second mesh-shaped silicone rubber intermediate layer, and a third silicone rubber layer which are provided in sequence, a tensile strength of the second mesh-shaped silicone rubber intermediate layer is greater than a tensile strength of the first silicone rubber layer and a tensile strength of the third silicone rubber layer.

3. The artificial dermal repair material according to claim 2, wherein the tensile strength of the first silicone rubber layer and/or the third silicone rubber layer is less than 2 MPa, the tensile strength of the second mesh-shaped silicone rubber intermediate layer is greater than 10 MPa.

4. The artificial dermal repair material according to claim 1, wherein the collagen complex layer is of a gradient porous structure.

5. The artificial dermal repair material according to claim 1, wherein the raw materials comprise 73% to 97% of the collagen, 2% to 15% of the glycosaminoglycan, and 1% to 12% of the antibacterial agent.

6. The artificial dermal repair material according to claim 5, wherein the raw materials comprise 78% to 93% of the collagen, 3% to 10% of the glycosaminoglycan, and 4% to 12% of the antibacterial agent.

7. The artificial dermal repair material according to claim 1, wherein at least one of the silicone rubber layer and the collagen complex layer is provided with a plurality of holes.

8. A method of preparing an artificial dermal repair material according to claim 1, comprising:
preparing a collagen complex layer, comprising:
dissolving glycosaminoglycan in a solvent to obtain a glycosaminoglycan solution, and dissolving collagen in a solvent to obtain a collagen solution;
mixing the glycosaminoglycan solution and the collagen solution, performing centrifugation and collecting a precipitate, and redissolving the precipitate by adding acetic acid to obtain a collagen-glycosaminoglycan complex solution; and
adding a cross-linking agent and an antibacterial agent into the collagen-glycosaminoglycan complex solution, performing freeze-drying, and then further performing a cross-linking reaction under a vacuum condition to obtain the collagen complex layer;
preparing a silicone rubber layer,
bonding the collagen complex layer and the silicone rubber layer, and
optionally preparing a hole on at least one of the collagen complex layer and the silicone rubber layer.

9. The method of preparing the artificial dermal repair material according to claim 8, wherein the preparing the silicone rubber layer comprises:
mixing and curing materials of a first silicone rubber to form a first silicone rubber layer;
mixing materials of a second silicone rubber to form a mixed solution, preparing a mesh-shaped layer on the first silicone rubber layer with the mixed solution by using a dispenser, and performing curing to form a second mesh-shaped silicone rubber intermediate layer; and
leveling materials of a third silicone rubber on a surface of the second mesh-shaped silicone rubber intermediate layer, and performing curing to form a third silicone rubber layer;
wherein a tensile strength of the materials of the second silicone rubber after film formation is greater than a tensile strength of the materials of the first silicone rubber and a tensile strength of the materials of the third silicone rubber after film formation.

10. The method of preparing the artificial dermal repair material according to claim 8, wherein the performing freeze-drying comprises: performing freezing under a low temperature environment, annealing for 5 min to 10 min, and then freezing under a low temperature environment, and then freeze-drying in a freeze dryer.

11. The method of preparing the artificial dermal repair material according to claim 8, wherein the cross-linking agent is one selected from the group consisting of glutaraldehyde, genipin, and carbodiimide.
